(19) United States
(12) Patent Application Publication
(10) Pub. No.: US 2026/0115377 A1
(43) Pub. Date: Apr. 30, 2026

(54) MEDICATION CONTAINER AND INFUSION SYSTEM HAVING SUCH A MEDICATION CONTAINER

(71) Applicant: ConnCons GmbH, Dresden (DE)

(72) Inventors: Christoph GOMMEL, Radebeul (DE); Jochen HAMPE, Dresden (DE); Carsten SCHURIG, Radebeul (DE)

(21) Appl. No.: 19/366,947

(22) Filed: Oct. 23, 2025

(30) Foreign Application Priority Data
Oct. 24, 2024 (EP) 24208684.1

(51) Int. Cl. A61M 5/142; A61B 90/98; A61J 1/10; A61M 39/10

(52) U.S. Cl. CPC A61M 5/142; A61B 90/98; A61J 1/10; A61M 39/10; A61M 2005/14208; A61M 2039/1044; A61M 2039/1083; A61M 2039/1088

(57) ABSTRACT

Medication container for use in infusion therapy and an infusion system incorporating such a medication container. Medication containers often flexible medication bags. The a medication container has a wall, which encloses an interior. A liquid to be administered to a patient can be held in this interior. The medication container also comprises at least one fluid coupling for connecting a fluid line for taking liquid from the medication container. The medication container comprises a first transmission device, wherein the first transmission device is configured for data communication with an information carrier applied on an outer side of a wall of the medication container.

### CROSS-REFERENCE TO RELATED APPLICATIONS

This application claims the priority of European patent application No. 24 208 684.1, filed Oct. 24, 2024, which is incorporated herein by reference in its entirety.

### TECHNICAL FIELD

The invention relates to a medication container in particular for use in infusion therapy, and to an infusion system having such a medication container.

### BACKGROUND

A medication container is used to hold medical liquids before they are administered to the patient. In particular, such a medication container may be formed as a medication bag, although a version with rigid walls is also possible.

Medical liquids in the context of the present disclosure can refer to any liquids used in the context of medical therapies, for example including nutrient solutions and blood products.

An intelligent medical fluid conduction and transfer system, and components therefor are described, for example, in US 2022/0273930 A1 and in WO 2021/023639 A1 from the same patent family.

Many therapy methods, for instance cancer therapy, employ personalized medical liquids that are individually adapted to the patient's needs. They are administered from medication containers of the generic type. The medication containers with such tailored medical liquids are conventionally labelled in order to avoid misallocations.

In order to ensure that no allocation errors occur in the course of a therapy, i.e. the use of a medication container that is not intended for the patient in question, it has already been proposed to provide medication containers with a memory so that information in the memory can be read by a control device of the infusion system.

US 2007/0145150 A1 describes a device for applying an RFID label carrier to an object. It is proposed that a holding part for the label carrier label be pivotably connected to an pasting part. The label can thus protrude and be pivoted or moved by a user toward a reader in order to be more easily read in different positions.

### SUMMARY

It is an object of an aspect of the invention to provide a medication container that further reduces the risk of a misallocation and/or to facilitate handling.

The claimed subject matter is defined in the independent claims. Further refinements are provided in the dependent claims.

According to an aspect of the present invention, it is suggested to provide a medication container, in particular for use in infusion therapy, which is configured in the manner described below. A medication container of the type according to an aspect of the present disclosure is usually technically uncomplicated and designed as a single-use or disposable product.

The medication container usually has a wall that encloses an interior in which the liquid to be administered can be held. The liquids may be, for example, sterile liquids, medicaments or nutrient solutions. This includes any liquids administered to the patient in the context of therapy. For example, this also includes plain water, solutions, blood constituents and blood.

The walls are usually primarily made of biocompatible and chemically stable plastic in order to ensure the sterility and integrity of the liquids. In particular, PVC, polyolefins, EVA or polyethylene may be envisaged for this. The walls may also be configured as metal walls or glass walls. The walls may additionally be provided with UV protection.

In particular, the medication container may be configured as a medication bag with a (geometrically) flexible bag wall. Such bags are often employed in infusion therapies. Nevertheless, aspects of the present disclosure also includes medication containers with rigid walls.

The medication container has at least one fluid coupling for attaching a fluid line for the purpose of taking liquid from the medication container, and in particular for the purpose of administering this liquid to a patient via an intravenous access.

The fluid coupling of the medication container may, for example, be configured in the manner of a Luer connector.

According to an aspect of the present disclosure, it is proposed that the medication container has a first transmission device for data communication with an information carrier applied on an outer side of a wall. In other words, the medication container comprises a first transmission device, wherein the first transmission device is adapted for data communication with an information carrier attached to an outside of the wall (which encloses the interior) of the medication container. The information carrier does not have to be part of the medication container. The medication container is configured such that the information carrier can be attached to the wall, and the first transmission device is adapted for data communication with the information carrier when the information carrier is attached to the outside of the wall enclosing the interior of the medication container. The information carrier can optionally be part of the medication container. This first transmission device is preferably provided on an anterior wall (front wall) of the medication container, in particular preferably approximately in the middle of the anterior wall.

During the intended use of the medication container, such an information carrier is applied on the outer side of the wall, in particular adhesively bonded there. The information carrier has an electronic memory, which is coupled to a second transmission device that is part of this information carrier.

In this way, the first and second transmission devices may be used in order to be able to read the electronic memory of the information carrier from the medication container.

The first transmission device and the second transmission device may be configured in a variety of ways in order to permit data communication between the medication container and the information carrier attached thereto. One simple possibility consists in configuring the first transmission device in the form of electrically conductive contact areas that are exposed on the outer side of the wall, optionally after removing a protective film. Correspondingly, electrical contact areas are also provided on the rear side of the information carrier, which bear on the contact areas of the medication bag after the information carrier has been applied.

Alternatively, the transmission device may also be configured as a capacitive or inductive transmission device.

A capacitive transmission device, which is introduced into the wall, comprises thin conductive capacitor plates, which are preferably enclosed inwards and outwards by films and are thereby electrically insulated outwards. The conductive capacitor plates act as electrodes that can generate and receive electric fields. Correspondingly, capacitor plates are also provided in the information carrier.

An inductive transmission device has a transmission coil, which is integrated into the wall. It is preferably a planar and in particular preferably flexible transmission coil, which is enclosed externally and internally by film layers of the wall. The transmission coil consists of one or more turns of conductive material, typically copper or aluminium. The covering film layer may for example be laminated on the wall, or applied there using an injection-moulded insert (in-mould labelling) or by ultrasonic or laser welding.

The transmission coil may in particular be configured in the form of a layer applied by printing or coating technology, or it may have been cut out from a flat material, in particular by means of laser cutting, and then integrated into the wall. It may also be produced by means of coating or printing. The transmission coil can be formed from a metallic material, which has preferably been brought into the form of the transmission coil by printing and coating or by laser cutting.

The transmission coil may be part of an NFC tag which, including an NFC chip, is applied on or introduced into the wall of the medication container.

Preferably, a support film is provided, which is applied on a wall of the medication container and to which the transmission coil or the capacitor plates of the first transmission device is/are applied. This support film is applied together with the transmission coil or the capacitor plates to a front wall of the medication container. The information carrier is applied, in particular adhesively bonded, externally on this film.

In the case of a medication bag, the aforementioned support film may have a size that corresponds to the size of the front wall on which it is applied, so that it forms a film that covers the entire front wall.

Preferably, the first transmission device is connected via signal lines to a data connector, so that the first transmission device can be connected via this data connector to an external control device. This data connector on the medication container allows the medication container to be coupled to a data line that leads to an external control device, so that this control device is connected via the data connector and via the first and second transmission devices to the memory of the information carrier, and can read it.

The data connector may in particular be integrated into the fluid coupling in a manner described in more detail below.

The aforementioned support film that supports at least the first transmission device may, together with the fluid coupling with an integrated data connector, form a preassembly unit which already forms a combined subunit before the support film is applied to the wall. This can facilitate assembly since all electrical lines between the first transmission device and the data connector are already part of the aforementioned preassembly unit, which obviates the electrical connecting between the data connector and the first transmission device when the support film is being applied.

In principle, it is possible that the medication container itself to comprises a control device in the form of an integrated circuit, which controls the data communication via the first transmission device and/or via the data connector. Nevertheless, a configuration is preferred in which the control device that controls the data communication via the first transmission device is not part of the medication container but is provided externally, for example integrated into a pump of the infusion system or coupled thereto. This external control device is connected via the data connector to the medication container and therefore to the first transmission device.

The signal lines of the medication container are preferably formed integrally, i.e. in one piece, with the transmission coil, the contact areas or the capacitor plates. In particular, the signal lines may be produced together with the first transmission device, which is formed as a transmission coil or capacitor plate, from the same metal foil. At least one signal line can be formed integrally with the transmission device configured as a transmission coil, in particular in the form of a composite produced from a metal foil composed of transmission coil turns and the signal line.

The data connector is preferably fixed in position with respect to the at least one fluid coupling so that, by attaching a connecting piece of a fluid line to the fluid coupling, a second data connector provided on the connecting piece is simultaneously connectable to the data connector of the medication bag.

In particular, the data connector may be integrated into the fluid coupling. In such a configuration, attaching the connecting piece to the fluid coupling also establishes the data connection. In respect of the configuration of the fluid coupling, reference is made to the document WO 2021/023639 A1 and its parallel publications, the disclosure of which in respect of the configurations of connecting pieces as a fluid coupling is included in the disclosure of the present disclosure by explicit reference.

The data connector may, in particular, be provided in the form of electrical contact areas at the fluid coupling. In the case of a transmission coil for data transmission at the fluid coupling, this transmission coil is preferably formed around the fluid line of the fluid coupling.

As described above, the medication container can optionally also comprise the information carrier. The information carrier can be attached to the wall of the medication container in the region of the first transmission device. The information carrier can comprise an electronic memory and a second transmission device, preferably with a transmission coil for inductive data transmission, so that the contents of the electronic memory can be read out via the first and second transmission devices.

The information carrier, which is configured in particular as an adhesive sticker and is equipped with the second transmission device, preferably not only contains information in the aforementioned electronic memory but is also provided with visually readable labelling, which is preferably provided here by printing.

In particular, a use is then envisioned in which the visually readable labelling and data in the memory at least partially match.

Such a configuration, in which both digital data of the information carrier and its labelling match, offers a significant advantage, such that discrepancies cannot occur when the information carrier is labelled and its memory is written in the same process. This ensures that the labelling and the information in the memory do not differ from one another due to a mistake.

The information carrier preferably has a multilayered structure, in particular comprising an inward-facing support film, a transmission coil as a second transmission device, and an outward-facing printing surface, which receives the labelling. In the prepared state, the medication container is provided with the information carrier, the latter being printed on and its electronic memory being written with information corresponding to the printed information.

The information carrier may in addition also have further electronic components. In particular, sensors that record the temperature and make it available for external reading via the first and second transmission devices may be envisioned here. The information carrier can for example comprise at least one sensor. The sensor can be read via the first and second transmission devices.

The medication container (for example the medication bag) with the first transmission device can be configured as an electronic reader for the information carrier to be attached to the outer sidewall of the medication container.

The first transmission device can be adapted for data communication with the information carrier applied on (i.e. to be applied on or to be attached to) the outside of the wall of the medication container, wherein the information carrier can be an RFID sticker. The first transmission device of the medication container can comprise a reading antenna for the RFID sticker.

Besides the medication container per se, aspects of the present disclosure also relate to an infusion system in which a medication container of the type described above is used. In respect of the structure of such an infusion system, reference is made to the document WO 2021/023639 A1 and its parallel publications, the disclosure of which in respect of the configuration of the infusion system is included in the disclosure of the present disclosure by explicit reference.

An infusion system according to an aspect of the present disclosure comprises a control device, in particular a control device integrated into a pump device or a control device connected to the pump device via a data line, as well as at least one medication container which is coupled via a fluid line.

The medication container is configured in the manner described above.

The aforementioned control device can be connected via a data line to the medication container, the data line preferably ending in a data connector that can be connected to a corresponding data connector of the medication container. The data line is preferably integrated into the fluid line. The data connectors belonging to the medication container and to the fluid line are preferably each integrated into fluid couplings, in particular into mating Luer connectors.

The control device is configured to read data on the information carrier applied to the medication container via the first transmission device of the medication container and the second transmission device of the information carrier.

These data may then be employed for validation with the aid of an existing medication plan. It is also possible for the medication plan itself to be stored in the memory of the information carrier and read by the control device.

### BRIEF DESCRIPTION OF THE DRAWINGS

Further advantages and aspects of the invention will become apparent from the claims and the following description of preferred exemplary embodiments of the invention, which are explained below with the aid of the drawings.

FIG. 1 shows an infusion system with medication containers according aspects of the present disclosure.

FIG. 2 shows a medication container according to an aspect of the present disclosure before the application of an information carrier.

FIG. 3 shows the information carrier.

FIG. 4 shows the medication container with the information carrier applied.

FIG. 5 shows the fluid coupling of the medication container and illustrates the data transmission from the medication container to an external control device.

### DETAILED DESCRIPTION

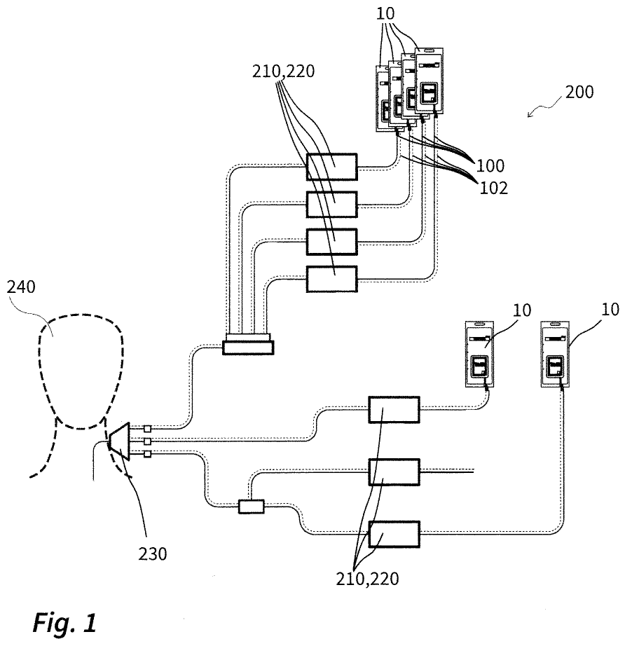

FIG. 1 shows the schematic representation of an infusion system 200 according to an aspect of the present disclosure. The infusion system 200 has a number of medication containers 10 in the form of (geometrically) flexible bags. The medication containers 10 are connected via fluid lines 100 to infusion pumps 210.

The infusion pumps 210 are configured to deliver the liquids from the medication containers 10 according to a medication plan to a central venous catheter 230 of a patient 240.

The infusion pumps 210 each have a control device 220 that controls the infusion pump. This control of the infusion pumps 210 in an infusion system 200 according to an aspect of the present disclosure also includes the data that come from the medication containers 10.

For this purpose, the medication containers 10 are each provided with a memory, as will be explained in more detail below.

The data transmission between the control devices 220 and the medication containers 10 takes place via data lines 102 that are integrated into the fluid lines 100. These data lines 102 are connected by a data line to the infusion pumps 210 and via the latter to the control devices 220.

At the medication container 10, the data lines 102 are connected via connecting pieces 30, 110 to the medication containers 10, which will be explained in more detail below.

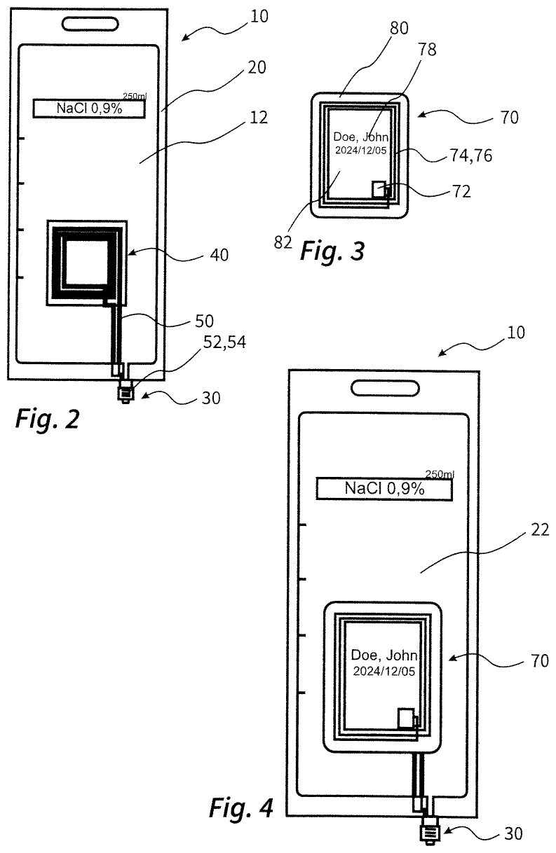

FIGS. 2 and 4 show a medication container 10 in the form of a flexible medication bag, and FIG. 3 shows an information carrier 70 applied thereon.

The medication container 10 that is shown in FIG. 2 does not comprise such an information carrier 70 yet. As can be seen from FIG. 2, the medication container 10 has walls 20, specifically an anterior wall 22 and a rear (posterior) wall, which enclose an interior 12 in which the medical liquid is held. At the lower end, the medication container 10 has a fluid coupling 30 for attaching a fluid line 100.

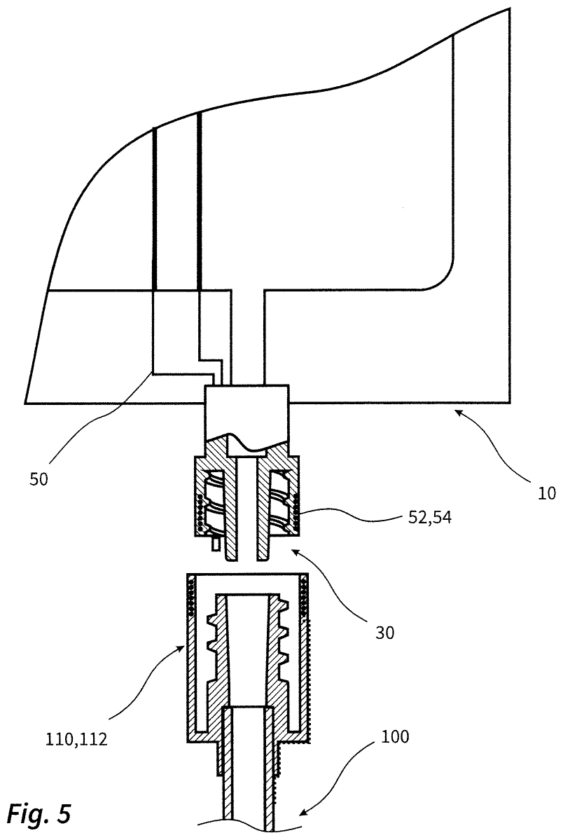

The medication container 10 furthermore has a first transmission device 40 in the form of a transmission coil on the anterior wall 22. This transmission coil is connected via signal lines 50 to a data connector 52, which likewise has a transmission coil 54 and which, in the present exemplary embodiment, is integrated into the fluid coupling 30. This will be explained in more detail below with the aid of FIG. 5.

The information carrier 70 shown in FIG. 3 is intended to be applied on the anterior wall 22 of the medication container 10. The information carrier 70 has an electronic memory 72, which is coupled to a transmission coil 76 of a second transmission device 74. The memory 72 and the transmission coil 76 may be elements of a commercially available NFC tag. Besides the electronic memory 72, the information carrier has a printing surface 82 that is intended to receive labelling or, in the present case, is already provided with labelling 78. In the present example, the labelling consists of the name of the patient as well as a date that indicates the shelf life of the content of the medication container 10. Other data which may be stored in the memory are an ID to which data are allocated in an external central memory. The memory may also contain proof of the authenticity of the medicament and/or a digital signature of the creator.

A peculiarity of the information carrier 70 is that it contains the same information, for example the expiry date in the present case, in electronic form in the memory 72 and in printed form in the form of the labelling 78. This information is preferably applied in an integral process. For example, a printer with an integrated NFC transmission device may be provided, which applies the labelling and stores the same data in the memory 72 in an integral process.

The information carrier 70 may have further electronic components. In particular, it may also comprise sensors, for example a temperature sensor, for monitoring with the aid of the latter whether the liquid is always kept at the target temperature. If it is also meant to be possible to acquire data by means of these sensors when the medication container 10 is not coupled to the data line 102 and is not supplied with power, this may be achieved by providing a battery and/or evaluation electronics on the information carrier or as part of the medication container.

FIG. 4 shows the medication container 10 with the information carrier 70 applied. In order to apply it, the information carrier 70 has a support film 80, on the rear side of which an adhesive surface may be provided. The information carrier 70 has been applied on the anterior wall 22 of the medication container 10 by means of this adhesive surface, the first transmission device 40 and its transmission coil 42 on the anterior wall 22 of the medication container 10 being covered by the information carrier 70 and in particular by its second transmission device 74 and its transmission coil 76.

By applying it in this way, the memory 72 can be read from the data connector 52. Sensor data may optionally also be read via the data connector 52.

FIG. 5 illustrates the way in which the data transmission takes place from the fluid line 100 to the medication container 10. Provided at the end of the fluid line 100 is a connecting piece 110, for example in the manner of a female Luer connector. Likewise provided on the medication container 10 is a connecting piece 30, which forms the fluid coupling 30 and which, for example, may be configured in the manner of a male Luer connector.

Both connecting pieces 30, 110 are equipped with an integrated data connector 52, 112. These data connectors 52, 112 are each equipped with a transmission coil 54, 114, so that inductive data transmission is enabled here. At the medication container 10, this transmission coil 54 is connected to the signal lines 50 which, as already described, are connected at the opposite end to the transmission coil 42. At the fluid line 100, the transmission coil 114 is connected to the data line 102 and therefore indirectly to the associated control devices 220.

In this way, it is possible for the control devices 220 to access the memory 72 via the transmission coils 54, 114 and via the transmission coil 42, 76 and electronically read the information that it contains. The control devices 220 may in this way, for example, determine whether the medication container 10 is actually intended for the patient 240 connected to the venous catheter 230. If this is not the case, delivery of liquid by means of the pump device 210 may be stopped.

At the same time, the labelling 78 and its creation as described above in an integral process with the storage of data in the memory 72 also ensure that the data of the information carrier 70 are consistent. This precludes the possibility of the labelling 78 giving the impression that the medication container is intended for the patient 240, but the data in the memory 72 contradicting this.

It is to be understood that the foregoing description is of one or more embodiments of the invention. The invention is not limited to the particular embodiment(s) disclosed herein, but rather is defined solely by the claims. Furthermore, the statements contained in the foregoing description relate to the disclosed embodiment(s) and are not to be construed as limitations on the scope of the invention or on the definition of terms used in the claims, except where a term or phrase is expressly defined above. Various other embodiments and various changes and modifications to the disclosed embodiment(s) will become apparent to those skilled in the art.

As used in this specification and claims, the terms “e.g.,” “for example,” “for instance,” “such as,” and “like” and the verbs “comprising,” “having,” “including,” and their other verb forms, when used in conjunction with a listing of one or more components or other items, are each to be construed as open-ended, meaning that the listing is not to be considered as excluding other, additional components or items. Other terms are to be construed using their broadest reasonable meaning unless they are used in a context that requires a different interpretation. In addition, the term “and/or” is to be construed as an inclusive OR. Therefore, for example, the phrase “A, B, and/or C” is to be interpreted as covering all of the following: “A”; “B”; “C”; “A and B”; “A and C”; “B and C”; and “A, B, and C.”

1. A medication container, for use in infusion therapy, having the following features:
a. the medication container has a wall, which encloses an interior, and
b. the medication container has at least one fluid coupling for attaching a fluid line for taking liquid from the medication container,
wherein:
c. the medication container has a first transmission device wherein the first transmission device is adapted for data communication with an information carrier applied on an outer side of the wall of the medication container.

2. The medication container according to claim 1, having the following further features:
a. the medication container is configured as a medication bag, and
b. the wall is configured as a flexible bag wall.

3. The medication container according to claim 1, having the following further features:
a. the first transmission device comprises electrically conductive contact areas on the outer side of the wall, or
b. the first transmission device is configured as a capacitive transmission device with embedded capacitor plates.

4. The medication container according to claim 1, having the following further feature:
a. the first transmission device has a transmission coil.

5. The medication container according to claim 4, having the following further feature:
a. a support film is provided, which is applied on a wall of the medication container and to which the transmission coil or capacitor plates is/are applied.

6. The medication container according to claim 1, having the following further feature:
a. the first transmission device is connected via signal lines to a data connector, via which the first transmission device can be connected to a control device.

7. The medication container according to claim 6, having the following further feature:
a. the data connector is fixed in position with respect to the at least one fluid coupling so that, by attaching a connecting piece of a fluid line to the fluid coupling, a second data connector provided on the connecting piece is simultaneously connectable to the data connector of the medication bag.

8. The medication container according to claim 7, having the following further feature:
a. the data connector is integrated into the fluid coupling, and
having the following further feature:
b. the data connector comprises electrical contact areas and/or a transmission coil.

9. The medication container according to claim 1, having the following further features:
a. the medication container comprises the information carrier, wherein the information carrier is applied on a wall of the medication container in a region of the first transmission device, and
b. the information carrier comprises an electronic memory and a second transmission device, so that the content of the electronic memory can be read via the first and second transmission devices.

10. The medication container according to claim 1, having the following further feature:
a. the information carrier has visually readable labelling,
having at least one of the following additional features:
b. the visually readable labelling and data in the memory at least partially match, and/or
c. the information carrier is formed as an adhesive sticker, and/or
d. the information carrier has a multilayered structure, in particular comprising an inward-facing support film, the transmission coil and an outward-facing printing surface.

11. The medication container according to claim 9, having the following further features:
a. the information carrier comprises at least one sensor, and
b. the sensor can be read via the first and second transmission devices.

12. The medication container according to claim 1, having the following further feature:
a. the first transmission device is provided on an anterior wall of the medication container.

13. An infusion system, having the following features:
a. the infusion system comprises a control device in particular a control device integrated into a pump device or a control device connected to the pump device via a data line, and
b. the infusion system comprises at least one medication container, which is coupled via a fluid line,
wherein
c. the medication container is configured according to claim 1, and
d. the control device is connected via a data line to the medication container, and
e. the control device is configured to read data on an information carrier applied to the medication bag via the first transmission device of the medication containerbag.

14. The medication container according to claim 4, having the following further feature:
b. the transmission coil is configured as a planar transmission coil, which lies in the plane of a wall of the medication container, and/or
c. the transmission coil is formed from a metallic material, which has been brought into the form of the transmission coil by printing and coating or by laser cutting.

15. The medication container according to claim 5, having any of the following further features:
b. the support film has a size that corresponds to the size of the wall on which it is applied, and/or
c. the support film together with the fluid coupling form a preassembly unit, which already forms a combined subunit before the support film is applied to the wall.

16. The medication container according to claim 6, having the following further feature:
b. the control device is not part of the medication container, and/or
c. at least one signal line is formed integrally with the transmission device configured as a transmission coil, in the form of a composite produced from a metal foil composed of transmission coil turns and the signal line.

17. The Medication container according to claim 12, wherein the first transmission device is provided in the middle of the anterior wall.

18. The medication container according to claim 1, wherein the medication container with the first transmission device is configured as an electronic reader for the information carrier attached to the outer sidewall of the medication container.

19. The medication container according to claim 1, having the following further features:
a. the first transmission device is adapted for data communication with the information carrier attached to the outside of the wall of the medication container, wherein the information carrier is an RFID sticker;
b. the first transmission device of the medication container comprises a reading antenna for the RFID sticker.
